(19) United States
(12) Patent Application Publication
(10) Pub. No.: US 2024/0209401 A1
(43) Pub. Date: Jun. 27, 2024

(54) CULTURE MEDIUM COMPOSITION FOR INCREASING GROWTH AND METABOLIC RATE OF ACETOGENIC STRAIN AND METHOD FOR CULTURING ACETOGENIC STRAIN USING THE SAME

(71) Applicant: GWANGJU INSTITUTE OF SCIENCE AND TECHNOLOGY, Gwangju (KR)

(72) Inventors: Ji Yeon KIM, Gwangju (KR); In Seop CHANG, Gwangju (KR); Mun Gyu LEE, Gwangju (KR); Nu Lee JANG, Gwangju (KR)

(21) Appl. No.: 17/915,190

(22) PCT Filed: May 12, 2022
(86) PCT No.: PCT/KR2022/006839

§ 371 Date: Sep. 28, 2022

(51) Int. Cl. C12P 7/62; C12N 1/20; C12N 1/32; C12R 1/01

(52) U.S. Cl. CPC C12P 7/62; C12N 1/205; C12N 1/32; C12R 2001/01

(57) ABSTRACT

An embodiment provides a culture medium composition for increasing the growth and metabolic rate of an acetogenic strain and a method for culturing an acetogen strain using the same. According to an embodiment, there is an effect of providing a culture medium composition capable of increasing the growth and metabolic rate of an acetogen by adding a C1 compound such as methanol that is a soluble substrate for the production of intermediates in a Wood-Ljungdahl pathway of acetogen, and a method for culturing an acetogenic strain using the same.

### TECHNICAL FIELD

The present invention relates to a culture medium composition for increasing the growth and metabolic rate of an acetogenic strain and a method for culturing an acetogen strain using the same, and more specifically, to a culture medium composition capable of increasing the growth and metabolic rate of an acetogenic strain by adding a C1 compound such as methanol as a soluble substrate for the production of intermediates in a Wood-Ljungdahl pathway of acetogen, and a method for culturing an acetogenic strain using the same.

### BACKGROUND ART

As environmental problems and energy depletion due to the use of petroleum-based compounds become increasingly serious, the world reduces carbon dioxide emissions to prevent global warming caused by the use of fossil fuels and expedites the development of various alternative energy sources in preparation for high oil prices.

Syngas that is a representative alternative energy source may be produced by reforming natural gas or gasifying solid raw materials such as coal, organic wastes, and biomass.

Such syngas has the following advantages as an alternative energy source.

First, since syngas can be converted from most hydrocarbons, risk of raw material exhaustion is low, price fluctuation is less than that of fossil fuels, and thus raw materials can be stably secured.

Second, in the case of syngas-based energy conversion, carbon dioxide is not emitted but is used. Therefore, there is little concern about environmental pollution caused by carbon dioxide generation.

Third, since main components of the syngas are hydrogen and carbon, the syngas can be converted into various high value-added products such as acetic acid, butyric acid, ethanol, and butanol. Therefore, the syngas has high utilization potential and is economical.

The syngas can be used through a biorefinery using microorganisms. Typically, anaerobic acetic acid-producing bacteria called acetogen are used. Acetogen fixes C1 gases such as carbon monoxide and carbon dioxide to acetyl-CoA through the Wood-Ljungdahl pathway and converts the microorganisms into organic acids such as acetic acid to obtain energy necessary for cell growth.

The syngas biorefinery using acetogen is directly affected by the growth rate and gas consumption rate of microorganisms, and thus there is a problem in that the syngas biorefinery has a relatively low reaction rate and production efficiency compared to a conventional chemical conversion process.

In particular, unlike the conventional fermentation technology based on soluble organic substances such as glucose and fructose, in the operation of a biological process using syngas that is a gaseous substrate, the solubility of the syngas in the aqueous phase, which is the culture condition of microorganisms, is very low. Therefore, there was a problem in that the growth and metabolic rate of microorganisms were further slow.

Therefore, it is very important to improve the growth and productivity of acetogen in order to increase process performance through the efficiency improvement of a biorefinery process, and furthermore, to develop an economical process system, and research on this is being actively conducted.

### DOCUMENT OF RELATED ART

(Patent Document 1) KR 10-2017-0076822 A1

### DISCLOSURE

### Technical Problem

The technical object to be achieved by the present invention is to solve the problems of the conventional art described above, and is to provide a strain culture medium composition and a method for culturing an acetogen strain using the same by improving the low consumption efficiency of a gas substrate of an acetogenic strain, and increasing an overall biorefinery process efficiency through the increase of the growth and metabolic rate of the strain.

The technical objects to be achieved by the present invention are not limited to the technical objects mentioned above, and other technical objects not mentioned will be clearly understood by those of ordinary skill in the art to which the present invention belongs from the description below.

### Technical Solution

In order to achieve the technical object, an embodiment of the present invention provides a culture medium composition for increasing a growth and metabolic rate of an acetogen strain.

The culture medium composition for increasing a growth and metabolic rate of an acetogen strain may contain a C1 compound.

The C1 compound may contain at least one selected from the group consisting of methanol, formic acid and formaldehyde.

The C1 compound may be methanol.

The methanol may be contained in a concentration of more than 0 M and 1.5 M or less with respect to a total medium composition.

The acetogenic strain may include the acetogenic strain capable of utilizing methanol.

The acetogen strain may include at least one selected from the group consisting of Eubacterium limosum, Clostridium autoethanogenum, Clostridium ljungdahlii, Clostridium carboxidivorans, Clostridium ragsdalei, Sporomusa ovata, Acetobacterium woodii, Acetobacterium dehalogenans, and Moorella thermoacetica.

The increase of the metabolic rate may be made through an increase in a consumption rate of a gas substrate of the acetogenic strain.

The gas substrate may include at least one of H₂ gas, CO gas, and CO₂ gas.

In order to achieve the technical object, another embodiment of the present invention provides a culture method for increasing a growth and metabolic rate of an acetogenic strain.

The culture method for increasing a growth and metabolic rate of an acetogenic strain may include a medium injection step of injecting an acetogenic strain culture medium composition comprising a C1 compound into a bioreactor; a strain inoculation step of inoculating the acetogenic strain into the culture medium composition; and a bioreactor driving step of culturing the acetogen strain by driving the bioreactor.

The C1 compound may be methanol.

### Advantageous Effects

According to an embodiment of the present invention, it is possible to provide a culture medium composition for increasing the growth and metabolic rate of an acetogenic strain capable of improving the efficiency of biorefinery process by adding a C1 compound such as methanol as a soluble substrate for the production of intermediates in a Wood-Ljungdahl pathway of acetogen so that the consumption rate of the gas substrate of the acetogenic strain is increased, and thus the overall growth and metabolic rate of the acetogen strain is increased.

In addition, according to an embodiment of the present invention, it is possible to provide a method for culturing an acetogen strain capable of improving the culture efficiency and product yield efficiency of acetogen by using the acetogen strain culture medium composition including the C1 compound.

The effects of the present invention are not limited to the above-described effects, and it should be understood to include all effects that can be inferred from the configuration of the invention described in the detailed description or claims of the present invention.

### DESCRIPTION OF DRAWINGS

FIG. 1 is a view showing a comparison of the mta operons of Eubacterium limosum strain and Acetobacterium woodii.

FIG. 2 is a view showing the Wood-Ljungdahl metabolic pathway of Eubacterium limosum strain and an estimation of the pathway that Eubacterium limosum strain uses methanol in the metabolic pathway.

FIG. 3 is a table showing the metabolic changes in Eubacterium limosum strain according to Comparative Examples 1 and 2 and Example 1 of the present invention by measuring protein expression.

FIG. 4 shows graphs of the growth rate, consumption rate of a substrate (CO, H₂) and product concentration of Eubacterium limosum strain according to Comparative Examples 1 and 2 and Examples 2 and 3 of the present invention measured over time.

FIG. 5 shows graphs of the growth rate, consumption rate of a substrate (CO, H₂, CO₂), methanol concentration and product concentration of Eubacterium limosum strain according to Comparative Example 3 and Examples 4 and 5 of the present invention measured over time.

### MODE FOR INVENTION

Hereinafter, the present invention will be described with reference to the accompanying drawings. However, the invention may be embodied in many different forms, and thus is not limited to the embodiments described herein. Further, in drawings, in order to clearly explain the present invention, parts irrelevant to the description are omitted, and similar reference numerals are attached to similar parts throughout the specification.

Throughout the specification, when it is said that a part is “connected (accessed, contacted, coupled)” with another part, this includes not only a case where it is “directly connected”, but also includes a case where another part is interposed therebetween. In addition, when a part “includes” a certain component, this means that other components may be further provided without excluding other components unless otherwise stated.

The terms used herein are used only to describe specific embodiments, and are not intended to limit the present invention. The singular expression includes the plural expression unless the context clearly dictates otherwise. In this specification, terms such as “comprises” or “have” are intended to designate that the features, numbers, steps, operations, components, parts, or combinations thereof described in the specification exist, and it should be understood that this does not preclude the possibility of addition or existence of one or more other features, numbers, steps, operations, components, parts, or combinations thereof.

Hereinafter, an embodiment of the present invention will be described in detail with reference to the accompanying drawings.

A culture medium composition for increasing the growth and metabolic rate of an acetogen strain according to an embodiment of the present invention will be described.

The culture medium composition for increasing the growth and metabolic rate of the acetogen strain may contain a C1 compound.

In this case, the C1 compound may contain at least one selected from the group consisting of methanol, formic acid and formaldehyde.

As described above, since the syngas biorefinery using acetogen is directly affected by the growth rate and gas consumption rate of microorganisms, and thus there is a problem in that the syngas biorefinery has a relatively low reaction rate and production efficiency compared to a conventional chemical conversion process. In particular, there is a problem that the growth and metabolic rate of microorganisms are slower due to the low solubility of the syngas component when the syngas is used as a raw material.

Based on this, the inventors of the present invention have come to the invention of a culture medium composition capable of increasing the overall metabolism by adding a C1 soluble compound that can be converted into an intermediate product of the Wood-Ljungdahl pathway in which acetogen fixes gaseous substrates such as carbon monoxide and carbon dioxide with acetyl-CoA so that the concentration of the metabolic intermediate can be increased.

Particularly, most preferably, the C1 soluble compound may be methanol.

The methanol is suitable as an additive for increasing the growth and metabolism of a strain in terms of cost without negatively affecting the growth of the strain when added to a strain culture medium.

In this case, the acetogen strain may contain the acetogen strain capable of utilizing the methanol.

Among several acetogen strains, some acetogens can metabolize methanol by methanol dehydrogenase or methyltransferase, and some use a metal transfer system for methanol utilization.

As such acetogen strains capable of utilizing methanol, there are, for example, Eubacterium limosum, Clostridium autoethanogenum, Clostridium ljungdahlii, Clostridium carboxidivorans, Clostridium ragsdalei, Sporomusa ovata, Acetobacterium woodii, Acetobacterium dehalogenans, Moorella thermoacetica.

In particular, Eubacterium limosum KIST612 has an operon (mta operon) encoding a methyltransferase similar to that of Acetobacterium woodii, which is a taxonomically closed acetogen.

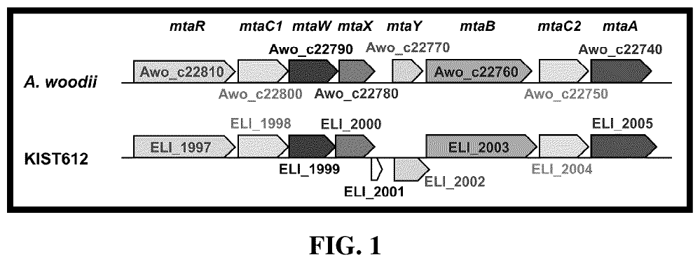

FIG. 1 is a view showing a comparison of the mta operons of Eubacterium limosum strain and Acetobacterium woodii.

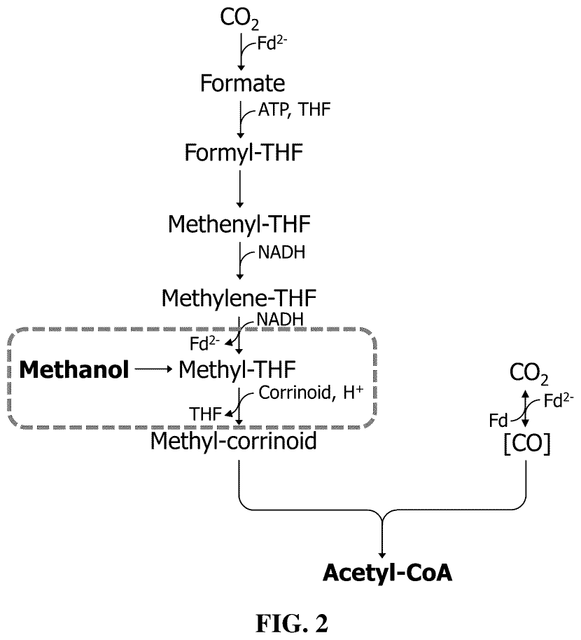

FIG. 2 is a view showing the Wood-Ljungdahl pathway of Eubacterium limosum strain and an estimation of the pathway that Eubacterium limosum strain uses methanol in the metabolic pathway.

Referring to FIG. 1, it can be confirmed that the Eubacterium limosum strain also has a methyltransferase capable of converting methanol into methyl-THF, similar to Acetobacterium woodii.

Referring to FIG. 2, it can be confirmed that the Eubacterium limosum uses the methyltransferase to convert methanol to methyl-THF, which is an intermediate metabolite of Wood-Ljungdahl pathway, thereby promoting autotrophic metabolism of the strain.

In this case, the gas substrate may contain at least one of H₂ gas, CO gas, and CO₂ gas.

The methanol preferably has a methanol concentration that can satisfy 0.008 g DCW/mmol MeOH or more in the entire medium.

In this case, the methanol may be contained in a concentration of more than 0 M and 1.5 M or less with respect to the total medium composition.

If the concentration of methanol exceeds 1.5 M, it is not preferable because growth inhibition occurs due to the addition of a high concentration of organic solvent.

Therefore, it is preferable that the methanol is contained in a concentration of more than 0 M and 1.5 M or less with respect to the total medium composition.

According to an embodiment of the present invention having the configuration characteristics as described above, the C1 compound, which is a soluble substrate convertible to an intermediate product of the Wood-Ljungdahl pathway is added to the culture medium, so that the consumption rate of the gas substrate of the acetogenic strain is increased. Accordingly, the overall growth and metabolic rate of the acetogenic strain is increased, so there is an effect of providing the culture medium composition for increasing the growth and metabolic rate of the acetogen strain capable of improving the efficiency of the biorefinery process.

A method for culturing an acetogenic strain according to another embodiment of the present invention will be described.

The method for culturing an acetogen strain may include a medium injection step of injecting the acetogen strain culture medium composition containing the C1 compound into a bioreactor; a strain inoculation step of inoculating the acetogenic strain into the culture medium composition; and a bioreactor driving step of culturing the acetogenic strain by driving the bioreactor.

In this case, the C1 compound may be methanol.

In the culture method, the cell concentration in a steady state (dilution rate of 0.018/h) in the presence of CO and CO₂ gas substrates without the addition of methanol may be maintained at 8.7 g/L.

In the culturing method under the conditions described below, when the methanol is added, the cell concentration in the steady state (dilution rate of 0.018/h) may be maintained at 17.4 g/L and the cell conversion yield may be 0.008 g DCW/mmol MeOH in the presence of CO and CO₂ gas substrates.

When the acetogen strain is batch-cultured by the culture method, the methanol may be contained in a concentration of 0 M to 1.5M in the culture medium composition in the medium injection step, and most preferably, the methanol may be contained in a concentration of 1.1 M.

When the acetogenic strain is cultured in a half batch by the above culture method, the acetogen strain culture medium composition containing methanol is injected into a reactor in the medium injection step, and then the strain is inoculated in the strain inoculation step. In the reactor driving step, after removing a portion of the culture solution in the reactor every predetermined time, the new culture solution containing methanol may be replenished by the same volume.

In this case, assuming that the removal rate of the culture solution in the reactor is 10%, the concentration in the culture solution must be adjusted so that 27.16 mmol of methanol can be added.

When the acetogenic strain is cultured in serial dilution by the above culture method, in the bioreactor driving step, the culture solution containing methanol is continuously supplied to the reactor, and the fermentation solution in the reactor is removed at the same flow rate.

In this case, assuming a dilution rate is 0.018/h, it may be operated in such a way that 19.56 mmol MeOH/L/hr of methanol is provided.

When the acetogen strain is cultured in a fed-batch manner by the above culture method, the acetogen strain culture medium composition containing methanol at a high concentration of 1 M or more in the bioreactor driving step is slowly supplied at a very slow flow rate, so it can be operated while minimizing a change of the water level in the reactor.

According to an embodiment of the present invention having the above configuration characteristics, there is an effect of providing a method of culturing an acetogen strain capable of improving acetogen culture efficiency and product yield efficiency by using the acetogenic strain culture medium composition containing the C1 compound.

Hereinafter, the present invention will be described in more detail through the Preparation Examples, Comparative Examples and Experimental Examples. However, the present invention is not limited to the following Preparation Examples and Experimental Examples.

### <Example 1> Culture of Eubacterium limosum KIST612 Strain by Adding Only Methanol without a Separate Gas Substrate

Eubacterium limosum KIST612 strain was cultured in a carbonate buffered basal medium (CBBM) containing 50 mM methanol. In this case, the CBBM was composed of the composition shown in Table 1 below.

TABLE 1

| CBBM | | | Vitamin solution (Final) | | Trace element solution | |
|---|---|---|---|---|---|---|
| | Conc. | | | Conc. | | Conc. |
| Components | (g/L) | | Components | (mg/L) | Components | (g/L) |
| NaCl | 0.9 | | Biotin | 2.0 | Nitrilotriaceticacid | 1.5 |
| MgSO₄7H₂O | 0.32 | | Folic acid | 2.0 | FeSO₄7H₂O | 0.1 |
| CaCl₂2H₂O | 0.2 | | Pyridoxine HCl | 10.0 | MnCl₂4H₂O | 0.1 |
| NH₄Cl | 1.0 | | Thiamine HCl | 5.0 | CoCl₂6H₂O | 0.17 |
| Yeast extract | 2.0 | | Riboflavin | 5.0 | ZnCl₂ | 0.1 |
| Vitamin | 10 | mL | Nicotinic acid | 5.0 | CaCl₂6H₂O | 0.1 |
| solution | | | | | | |
| Trace element | 10 | mL | Pantothenic acid | 5.0 | CuCl₂2H₂O | 0.02 |
| sol. | | | | | | |
| Sodium | 2.1 | | Cyanocobalamine | 0.1 | H₃BO₃ | 0.01 |
| bicarbonate | | | | | | |
| 1MK₂HPO₄ | 10 | mL | r-aminobenzoic | 5.0 | Na₂MoO₄ | 0.01 |
| | | | acid | | | |
| L-cysteine HCl | 0.5 | | Lipoic acid | 5.0 | Na₂SeO₃ | 0.017 |
| 0.1% (w/v) | 200 | μl | | | NiSO₄6H₂O | 0.026 |
| resazurin | | | | | | |
| CH₃OH | 0-50 | mM | | | NaCl | 1.0 |

### <Example 2> Culture of Eubacterium limosum KIST612 Strain by Adding MeOH Under CO/CO₂ Condition

Using CO/CO₂ as an energy and electron sources, the Eubacterium limosum KIST612 strain was cultured in the same carbonate buffered basal medium (CBBM) as CBBM of Example 1, containing 50 mM methanol. In this case, the CO and CO₂ were added in a ratio of 8:2.

### <Example 3> Culture of Eubacterium limosum KIST612 Strain by Adding MeOH Under H₂/CO₂ Condition

Using H₂/CO₂ as an energy and electron sources, the Eubacterium limosum KIST612 strain was cultured in the same carbonate buffered basal medium (CBBM) as CBBM of Example 1, containing 50 mM methanol. In this case, the H₂ and CO₂ were added in a ratio of 8:2.

### <Example 4> Culture of Eubacterium limosum KIST612 Strain by Adding MeOH Under H₂/CO/CO₂ Condition

Using H₂/CO/CO₂ as an energy and electron sources, the Eubacterium limosum KIST612 strain was cultured in the same carbonate buffered basal medium (CBBM) as CBBM of Example 1, containing 15 mM methanol. In this case, H₂, CO and CO₂ were added in a ratio of 4:5:1, respectively.

### <Example 5> Culture of Eubacterium limosum KIST612 Strain by Adding MeOH Under H₂/CO/CO₂ Condition

The Eubacterium limosum KIST612 strain was cultured under the same process condition as in Example 4, except that it contained 30 mM methanol.

### <Comparative Example 1> Culture of Eubacterium limosum KIST612 Strain without MeOH Addition Under CO/CO₂ Condition

The Eubacterium limosum KIST612 strain was cultured under the same process condition as in Example 1, except that, unlike Example 1, methanol was not additionally added.

### <Comparative Example 2> Culture of Eubacterium limosum KIST612 Strain without MeOH Addition Under H₂/CO₂ Condition

The Eubacterium limosum KIST612 strain was cultured under the same process condition as in Example 2, except that, unlike Example 2, methanol was not additionally added.

### <Comparative Example 3> Culture of Eubacterium limosum KIST612 Strain without MeOH Addition Under H₂/CO/CO₂ Condition

The Eubacterium limosum KIST612 strain was cultured under the same process condition as in Example 3, except that, unlike Example 3, methanol was not additionally added.

### <Experimental Example 1> Experiment for Analyzing the Metabolic Change of an Acetogen Strain by Methanol Addition Under CO/CO₂ or H₂/CO₂ Condition

Experiment was conducted to measure the metabolic change, growth rate, consumption rate of substrate (CO, H₂), product concentration and product production rate of the KIST612 strain cultured in Comparative Examples 1 and 2 and Examples 1 to 3.

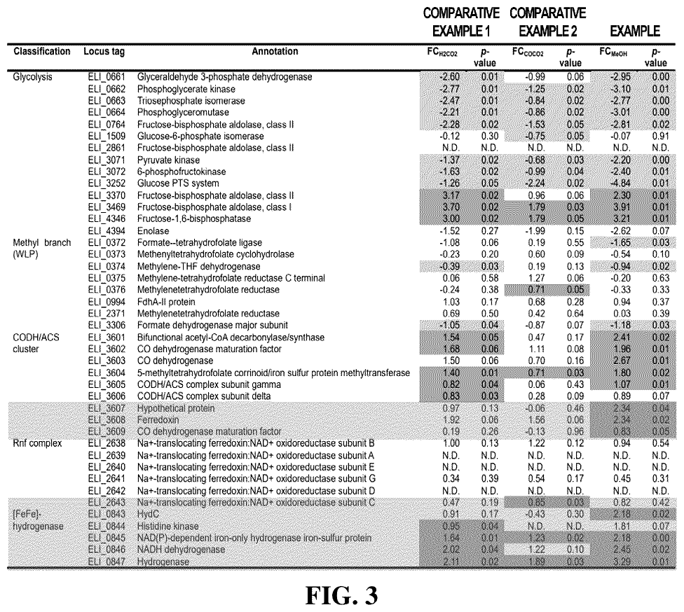

FIG. 3 is a table showing the metabolic changes of the KIST612 strain according to Comparative Examples 1 and 2 and Example 1 of the present invention by measuring protein expression.

In FIG. 3, green parts show downregulated proteins, and red parts show upregulated proteins.

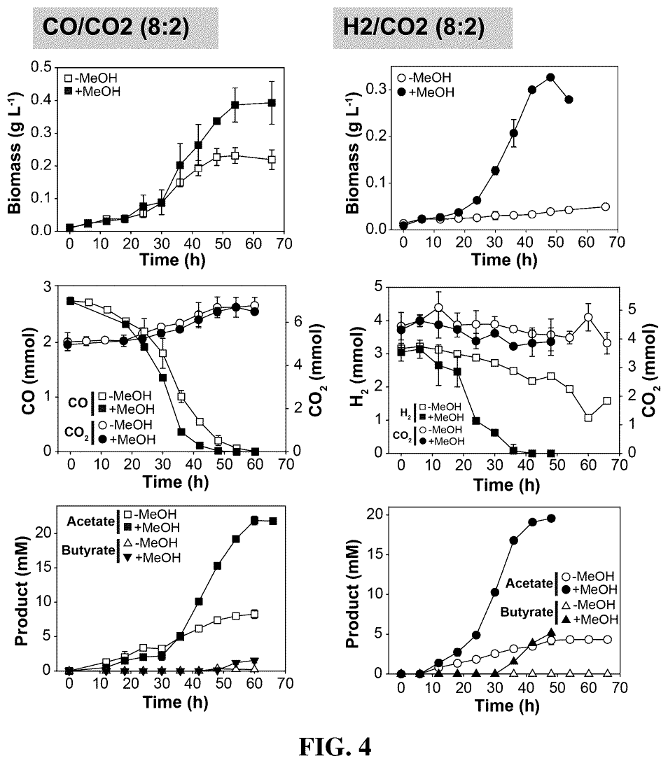

FIG. 4 shows graphs of the growth rate, consumption rate of substrate (CO, H₂) and product concentration of the KIST612 strain according to Comparative Examples 1 and 2 and Examples 2 and 3 of the present invention measured over time.

Referring to FIGS. 3 and 4, the KIST612 was grown for 48 hours at growth rates of 0.03±0.02/h and 0.12±0.01/h, respectively, under the conditions of CO/CO₂ of Example 2 or H₂/CO₂ of Example 3. Under these conditions, the consumption rate of CO or H₂ were 2.0±0.2 mmol g/cell h and 3.1±1.5 mmol g/cell h, respectively.

Referring to Example 3, it can be confirmed that a more significant change is observed in the physiological properties of the KIST612 under H₂/CO₂ condition. In the presence of methanol of Example 3, the specific growth rate was 0.12±0.01/h and the consumption rate of H₂ was 8.3±5.4 mmol g/cell h, which were 4.0 times and 2.7 times higher than those of Comparative Example 2 in which methanol was not added, respectively. Therefore, it can be confirmed that the addition of methanol can improve the utilization rate of H₂/CO₂, especially in the KIST612.

### <Experimental Example 2> Experiment for Analyzing the Consumption Rate of Substrate and Metabolic Change of an Acetogen Strain by Methanol Addition Under CO/H₂/CO₂ Condition

Experiment was conducted to measure the growth rate, consumption rate of substrate (CO, H₂, CO₂), product concentration and product production rate of the KIST612 strain cultured in Comparative Example 3 and Examples 4 and 5.

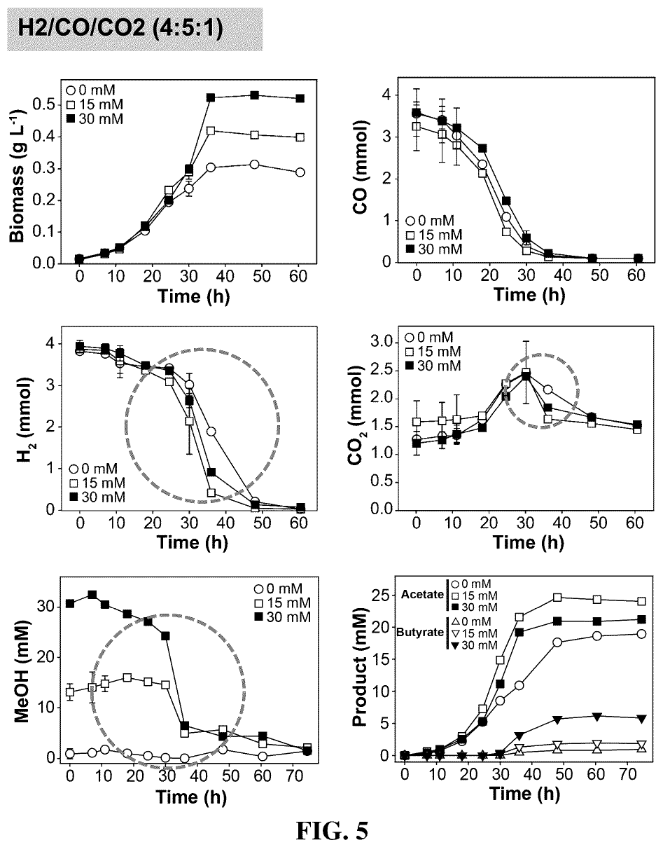

FIG. 5 shows graphs of the growth rate, consumption rate of substrate (CO, H₂, CO₂), methanol concentration and product concentration of the KIST612 strain according to Comparative Example 3 and Examples 4 and 5 of the present invention measured over time.

Referring to FIG. 5, in Examples 4 and 5 in which methanol was added, the growth rate of cells was faster, and in particular, it was confirmed that H₂ was consumed more rapidly than in Comparative Example 3. Further, it was confirmed that the product production rate was also increased in the presence of methanol. In particular, the sharp decrease in the methanol concentration suggests that the strain can use methanol very easily if only the growth conditions of the strain are properly adjusted, unlike the gas substrate.

The description of the present invention described above is for illustration, and those of ordinary skill in the art to which the present invention pertains can understand that it can be easily modified into other specific forms without changing the technical spirit or essential features of the present invention. Therefore, it should be understood that the embodiments described above are illustrative in all respects and not restrictive. For example, each component described as a single type may be implemented in a distributed form, and likewise components described as distributed may also be implemented in a combined form.

The scope of the present invention is indicated by the following claims, and all changes or modifications derived from the meaning and scope of the claims and their equivalents should be construed as being included in the scope of the present invention.

1. A culture medium composition for increasing a growth and metabolic rate of an acetogen strain, comprising a C1 compound.

2. The culture medium composition according to claim 1, wherein the C1 compound comprises at least one selected from the group consisting of formic acid and formaldehyde.

3. The culture medium composition according to claim 1, wherein the C1 compound is methanol.

4. The culture medium composition according to claim 3, wherein the methanol is comprised in a concentration of more than 0 M and 1.5 M or less with respect to a total medium composition.

5. The culture medium composition according to claim 1, wherein the acetogenic strain comprises the acetogenic strain capable of utilizing methanol.

6. The culture medium composition according to claim 1, wherein the acetogenic strain comprises at least one selected from the group consisting of Eubacterium limosum, Clostridium autoethanogenum, Clostridium ljungdahlii, Clostridium carboxidivorans, Clostridium ragsdalei, Sporomusa ovata, Acetobacterium woodii, Acetobacterium dehalogenans, and Moorella thermoacetica.

7. The culture medium composition according to claim 1, wherein the increase of the metabolic rate is made through an increase in a consumption rate of a gas substrate of the acetogenic strain.

8. The culture medium composition according to claim 7, wherein the gas substrate comprises at least one of H₂ gas, CO gas, and CO₂ gas.

9. A method for culturing an acetogenic strain, comprising:
a medium injection step of injecting an acetogen strain culture medium composition comprising a C1 compound into a bioreactor;
a strain inoculation step of inoculating the acetogenic strain into the culture medium composition; and
a bioreactor driving step of culturing the acetogen strain by driving the bioreactor.

10. The method for culturing an acetogen strain according to claim 9, wherein the C1 compound comprises at least one selected from the group consisting of formic acid and formaldehyde.

11. The method for culturing an acetogen strain according to claim 9, wherein the C1 compound is methanol.
